(19) United States
(12) Patent Application Publication
(10) Pub. No.: US 2006/0009539 A1
(43) Pub. Date: Jan. 12, 2006

(54) Maleimide-based radiation curable compositions

(76) Inventors: Donald E. Herr, Doylestown, PA (US); Darwin Scott Bull, Hillsborough, NJ (US)

Correspondence Address: NATIONAL STARCH AND CHEMICAL COMPANY, P.O. BOX 6500, BRIDGEWATER, NJ, 08807-3300, US

(21) Appl. No.: 10/889,202

(22) Filed: Jul. 12, 2004

(51) Int. Cl. C07D 209/48

(52) U.S. Cl. 522/176

(57) ABSTRACT

The invention is directed to aromatic maleimides as photocrosslinkers for unsaturated compositions. The maleimides utilized are multifunctional, and are attached to a polymeric backbone. As such, they are polymeric or polymer-bound photoinitiators/photocrosslinkers. The polymeric maleimides are necessarily aromatic, but may or may not exhibit substituents at the 3- and 4-position of the maleimide ring. These maleimide photocrosslinkers may be used alone or in conjunction with a photosensitizer to effectively crosslink unsaturated materials.

### FIELD OF THE INVENTION

This invention relates to the use of polymeric photoinitiators and photocrosslinkers that are functionalized with aromatic maleimide groups. These polymeric photoinitiators can be utilized to photocure unsaturated materials. Such aromatic maleimide-functional photoinitiators/crosslinkers are often used in conjunction with a photosensitizer.

### BACKGROUND

Radiation curing is a well-established means to quickly and efficiently build polymer molecular weight or create crosslinked systems. The general benefits of light-induced chemistry and crosslinking has been widely discussed in the literature. There are several common issues which must be addressed to varying degrees when utilizing light curable systems. Among the most important of these is minimizing (or eliminating) extractable or volatile photo by-products. Such by-products frequently exhibit odor or present toxicological issues if they are eventually extracted, or otherwise removed from, from the cured polymer matrix. A very common source of such odorous or extractable by-products is low molecular weight photoinitiator fragments or photoproducts. This is true of both the α-cleavage (“Type I”) and hydrogen-abstraction (“Type II”) classes of photoinitiators. As such, much historical and contemporary research in the area of radiation curable systems has focussed on polymeric or polymerizeable photoinitiators that exhibit reduced levels of problematic photo by-products.

A basic approach to reducing/eliminating small molecule photo by-products is to utilize photoinitiators that can copolymerize with the developing polymer matrix as radiation curing occurs. This is basically achieved by functionalizing the photoinitiator chromophore with a moiety that will react into the developing polymer matrix formed upon irradiation. While this approach will frequently reduce the levels of photoinitiator-derived extractables, any copolymerizeable photoinitiator or photoinitiator species that do not react with the developing polymer network may still eventually be removed from the cured material. For example, if Type I (α-cleavage) systems are so functionalized, both fragments formed via photocleavage need to react into the curing matrix to eliminate all small molecule photo by-products. Most so functionalized Type I photoinitiators know in the prior art exhibit a reactive/copolymerizeable moiety on only one of the fragments eventually formed upon α-cleavage, and as such half of the photoinitiator fragments formed are unbound and mobile after irradiation. They may be extracted or volatilized as usual. If Type II (H-abstraction) systems are utilized, both the aromatic ketone and any necessary co-reagents (“synergists”) need to crosslink into the growing polymer in order to eliminate extractable by-products. Naturally, any functionalized photoinitiator molecules or functionalized fragments that do not effectively copolymerize with the developing light cured matrix will remain unbound and mobile as well. While this “reactive small molecule photoinitiator” is a valid, and often satisfactory, approach to reducing photoinitiator-derived extractable components, better systems are often required for certain types of products. Examples include adhesives, coatings, or inks for use in direct food or skin contact applications.

For such demanding applications, further measures must be taken to ensure photoinitiator-derived species cannot be extracted from the cured product or become volatile photoproducts. An advanced option is the use of high molecular weight or polymeric photoinitiators. In particular, polymeric photoinitiators that do not function through cleavage photochemistry provide the possibility of completely odor- and extractable-free radiation curable systems. If such polymeric photoinitiators are multifunctional, they may also function as crosslinkers for the system and contribute favorably to its overall physical or mechanical properties. In general, a non-fragmenting photoinitiator chromophore can be incorporated into a polymeric material either as a pendant group, in the polymer backbone, or at the polymer termini as endgroups. The polymeric photoinitiators of this invention contain either pendant or terminal maleimide functionality. They are often preferentially used in conjunction with a photosensitizer, most often a triplet photosensitizer with a triplet energy of more than 57 kcal/mol.

### SUMMARY OF THE INVENTION

The utility of aromatic maleimides is often discounted due to their slightly different photochemical behavior relative to aliphatic analogs. Aliphatic maleimides are significantly more difficult to synthesize than aromatic maleimides, often requiring unusual or expensive dehydrating agents in order to close the amic acid ring to form the maleimide functionality. Conversely, the synthesis of aromatic maleimides is often cheap and high yield. As such, it would be useful to utilize the more practical/economical aromatic maleimides as photocrosslinkers whenever possible. As described in the background section, if maleimides are to be utilized in photocurable systems wherein low odor and low extractables are necessary, it is desireable that they be present in a polymeric or polymer-bound form.

Thus, the present invention discloses the use of aromatic maleimides as photocrosslinkers for unsaturated compositions. The maleimides utilized are multifunctional, and are attached to a polymeric backbone. As such, they are polymeric or polymer-bound photoinitiators/photocrosslinkers. The polymeric maleimides are necessarily aromatic, but may or may not exhibit substituents at the 3- and 4-position of the maleimide ring. These maleimide photocrosslinkers may be used alone or in conjunction with a photosensitizer to effectively crosslink unsaturated materials. Preferred is the radiation crosslinking of unsaturated polyolefins with the polymer-bound maleimides of the present invention.

### DETAILED DESCRIPTION OF THE INVENTION

The inventive radiation curable composition comprises three basic components:
a) an unsaturated small molecule or polymer
b) a polymeric aromatic maleimide compound, and
c) optionally, a photosensitizer.
   Radiation is defined as non-ionizing electromagnetic radiation (“actinic radiation”). Often, this radiation exhibits energy that places it in the ultraviolet (UV) or visible wavelengths.

The unsaturated compound has no particular limitation. In general, it will be any compound possessing double bonds that are susceptible to UV induced crosslinking or photoreaction. The unsaturated material may be a low molecular weight material (“small molecule”) or polymeric in nature, depending on the end use application. The double bonds in this compound may react through any mechanism, but are often those that undergo radical polymerization/oligomerization or those that readily undergo [2+2] cycloaddition photcrosslinking. No particular radiation crosslinking mechanism is specifically required or implied. In many cases multiple crosslinking mechanisms are likely. The unsaturated component may be a blend of different olefins as well. Often, the preferred unsaturated compound is a styrene-butadiene-styrene or styrene-isoprene-styrene block copolymer.

The polymeric aromatic maleimide compound generally conforms to the following structure: 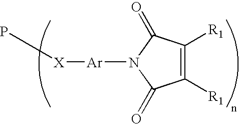
wherein R₁ is independently H, alkyl, cycloalkyl, or aryl,
Ar is an aromatic ring that may contain heteroatoms,
X is O, S, NH, C(O), O—C(O)—, —C(O)—O,
P is a polymeric backbone comprising alkyl, cycloalkyl, or aromatic groups which may contain heteroatoms
   and n=2-100.

The exact form of the polymeric aromatic maleimide is chosen to be chemically and morphologically compatible with the resin system into which it is blended as a photocrossliker/photoinitiator. The aromatic maleimide groups may be pendant or terminal to the main polymer chain. The polymer backbone, P, may take on any architecture known to those skilled in the art, such as linear, radial, dendrimeric, or hyperbranched. Often, the preferred polymer backbone P is poly(tetramethylene oxide), the preferred linking group X is —O—C(O)—, the preferred disubstituted Ar group is simply C₆H₄ aryl, and the preferred R₁ groups are H.

The optional photosensitizer is any small molecule or polymeric chromophore which can function to transfer absorbed energy to the maleimide compound. The general principles for selecting an appropriate photosensitizer are known to those skilled in the art. The photosensitizer is often a compound with a red-shifted UV absorbance relative to the aromatic maleimide material. The photosensitizer will typically be a triplet photosensitizer possessing a triplet state with energy greater than that of the excited triplet state of the maleimide (ca. 57 kcal/mol). Often the preferred photosensitizer is a small molecule or polymeric thioxanthone derivative.

lit is often desirable to utilize polymeric unsaturated materials (a), polymeric aromatic maleimide crosslinkers (b), and polymeric or innocuous photosensitizers (c). Thus, using the inventive materials to be further described hereafter and in the example section, one can formulate a radiation curable system that exhibits essentially none of the odorous or extractable by-products encountered using photoinitiators and crosslinkers known in the prior art. If all of the basic components of the invention are polymeric it is, in principle, possible to develop light curable materials with zero extractable or volatile/odorous components. Such low/no extractable type systems are extremely valuable in common radiation cure application areas such as coatings, adhesives, sealants, and inks. The current aromatic maleimide-containing radiation curable compositions can be used for all of these application areas through proper formulation techniques known to those skilled in the art of developing light curable products.

The basic components of the inventive composition can be combined with a variety of other components in order to produce a fully formulated product. If appropriate, inorganic or organic filler components may be present. Such fillers include, but are not limited to, silica, alumina, titanium dioxide, calcium carbonate, boron nitride, aluminum nitride, silver, copper, gold, talc and mixtures thereof. If appropriate, non-reactive components may also be present. Such components might include plasticizers, tackifiers, or other diluents. Reactive components that cure through a mechanism other than that induced by the aromatic maleimide component may also be present. Such components might include, but are not limited to, epoxy resins, cyanate ester resins, isocyanate-functional materials, or silicone components which cure through either condensation or addition cure mechanisms.

The above basic description is further delineated through the following non-limiting examples.

### EXAMPLES

Bismaleimides (BMI) were prepared from commercial polymeric arylamines (Air Products Versalink® Oligomeric Diamines P-250, P-650, and P-1000) as described in U.S. Pat. No. 4,745,197. These polymeric bismaleimides were then evaluated as UV crosslinkers in styrene-isoprene-styrene (SIS) triblock polymer systems (Kraton® D1165). The test formulations were based on 50 wt % SIS, 50% (nominal) Kaydol® oil, and polymeric BMI, isopropylthioxanthone (ITX), and titanium dioxide (Dupont Ti-Pure® R-104) as indicated. The method of evaluation involved dissolving the formulation components in toluene and casting films onto a release liner. Upon drying, the films were irradiated on a Fusion UV® conveyor line, removed from the release liner, and placed in toluene to dissolve any uncrosslinked polymer. The solutions were then filtered through tarred filter paper. The filter paper with the insoluble polymer fraction was then dried. Gel contents are reported as the percentage of residual undissolved polymer mass relative to the initial polymer mass. Control films that were irradiated in the absence of the bismaleimide resins with or without isopropylthioxanthone exhibited gel contents of 0-6%. Curing efficacy of specific formulations is described in the following examples.

### Examples 1-4

In the following examples, a bismaleimide based on Versalink® P-250 was used as the UV crosslinker with or without isopropylthioxanthone as a photosensitizer. In addition, curing in the presence or absence of TiO₂ was evaluated. Films of 4-5 mil dry thickness were cured using a D-lamp at a conveyor speed of 20 feet/min, which corresponded to energy densities of 1730 mJ/cm² UV-A, 750 mJ/cm² UV-B, and 78 mJ/cm² UV-C. Component percentages are given as weight % of the full formulation as shown in Table 1

TABLE 1

| Example | BMI (%) | ITX (%) | TiO₂ (%) | Gel Content (%) |
|---|---|---|---|---|
| 1 | 5 | — | — | 2 |
| 2 | 5 | 0.5 | — | 91 |
| 3 | 5 | — | 4 | 4 |
| 4 | 5 | 0.5 | 4 | 70 |

### Examples 5-7

In the following examples, a bismaleimide based on Versalink® P-250 was used as the UV crosslinker with isopropylthioxanthone as a photosensitizer. In addition, TiO₂ was used in all cases. In these examples, films of 3 mil dry thickness were cured using a D-lamp at a conveyor speed of 30 feet/min, which corresponded to energy densities of 1090 mJ/cm² UV-A, 445 mJ/cm² UV-B, and 46 mJ/cm² UV-C. Component percentages are given as weight % of the full formulation and are shown in Table 2.

TABLE 2

| Example | BMI (%) | ITX (%) | TiO₂ (%) | Gel Content (%) |
|---|---|---|---|---|
| 5 | 5 | 0.5 | 2 | 57 |
| 6 | 3 | 0.3 | 2 | 61 |
| 7 | 1 | 0.1 | 2 | 3 |

### Examples 8-12

In the following examples, a bismaleimide based on Versalink® P-650 was used as the UV crosslinker with or without isopropylthioxanthone as a photosensitizer. In addition, curing in the presence or absence of TiO₂ was evaluated. Films of 4-5 mil dry thickness were cured using a D-lamp at a conveyor speed of 20 feet/min, which corresponded to energy densities of 1730 mJ/cm² UV-A, 750 mJ/cm² UV-B, and 78 mJ/cm² UV-C. Component percentages are given as weight % of the full formulation and are shown in Table 3.

TABLE 3

| Example | BMI (%) | ITX (%) | TiO₂ (%) | Gel Content (%) |
|---|---|---|---|---|
| 8 | 5 | — | — | 2 |
| 9 | 5 | 0.5 | — | 100 |
| 10 | 5 | — | 4 | 8 |
| 11 | 5 | 0.5 | 4 | 82 |
| 12 | 5 | 0.5 | 2 | 90 |

### Examples 13-15

In the following examples, a bismaleimide based on Versalink® P-650 was used as the UV crosslinker with isopropylthioxanthone as a photosensitizer. In addition, TiO₂ was used in all cases. In these examples, films of 3 mil dry thickness were cured using a D-lamp at a conveyor speed of 30 feet/min, which corresponded to energy densities of 1090 mJ/cm² UV-A, 445 mJ/cm² UV-B, and 46 mJ/cm² UV-C. Component percentages are given as weight % of the full formulation in Table 4.

TABLE 4

| Example | BMI (%) | ITX (%) | TiO₂ (%) | Gel Content (%) |
|---|---|---|---|---|
| 13 | 5 | 0.5 | 2 | 94 |
| 14 | 3 | 0.3 | 2 | 68 |
| 15 | 1 | 0.1 | 2 | 14 |

### Examples 16-17

In the following examples, a bismaleimide based on Versalink® P-1000 was used as the UV crosslinker with or without isopropylthioxanthone as a photosensitizer. These formulations contained 2% TiO₂. Films of 4-5 mil dry thickness were cured using a D-lamp at a conveyor speed of 20 feet/min, which corresponded to energy densities of 1730 mJ/cm² UV-A, 750 mJ/cm² UV-B, and 78 mJ/cm² UV-C. Component percentages are given as weight % of the full formulation in Table 5.

TABLE 5

| Example | BMI (%) | ITX (%) | TiO₂ (%) | Gel Content (%) |
|---|---|---|---|---|
| 16 | 5 | — | 2 | 5 |
| 17 | 5 | 0.5 | 2 | 94 |

### Examples 18-27

In the following examples, a bismaleimide based on Versalink® P-1000 was used as the UV crosslinker with isopropylthioxanthone as a photosensitizer. In addition, TiO₂ was used in all cases. In these examples, films of 3 mil dry thickness were cured using a D-lamp at a conveyor speed of 30 feet/min, which corresponded to energy densities of 1090 mJ/cm² UV-A, 445 mJ/cm² UV-B, and 46 mJ/cm² UV-C. Component percentages are given as weight % of the full formulation in Table 6.

TABLE 6

| Example | BMI (%) | ITX (%) | TiO₂ (%) | Gel Content (%) |
|---|---|---|---|---|
| 18 | 5 | 0.5 | 2 | 85 |
| 19 | 3 | 0.5 | 2 | 68 |
| 20 | 3 | 0.3 | 2 | 73 |
| 21 | 3 | 0.1 | 2 | 28 |
| 22 | 2 | 2 | 2 | 67 |
| 23 | 2 | 1 | 2 | 80 |
| 24 | 2 | 0.5 | 2 | 74 |
| 25 | 2 | 0.3 | 2 | 58 |
| 26 | 2 | 0.1 | 2 | 20 |
| 27 | 1 | 0.1 | 2 | 3 |

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A radiation curable composition comprising
a) an unsaturated small molecule or polymer
b) an aromatic maleimide compound having the structure: 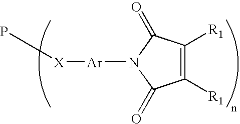
wherein R₁ is independently H, alkyl, cycloalkyl, or aryl,
Ar is an aromatic ring that may contain heteroatoms,
X is O, S, NH, C(O), O—C(O)—, —C(O)—O,
P is a polymeric backbone comprising alkyl, cycloalkyl, or aromatic groups which may contain heteroatoms
and n=2-100 and
c) optionally, a photosensitizer.

2. The composition of claim 1 wherein R₁ is H, Ar is a benzene aromatic ring, x is —O—C(O)—, and P is a polyether backbone.

3. The composition of claim 1 wherein the unsaturated component a) comprises an unsaturated polyolefin.

4. The composition of claim 3 wherein the unsaturated polyolefin is a styrene-butadiene-styrene or styrene-isoprene-styrene block copolymer.

5. The composition of claim 2 wherein the photosensitizer c) is isopropylthioxanthone.

6. The composition of claim 1 further comprising one or more filler from the group consisting of silica, alumina, titanium dioxide, calcium carbonate, boron nitride, aluminum nitride, silver, copper, gold, talc and mixtures thereof.
